(19) United States
(12) Patent Application Publication
(10) Pub. No.: US 2021/0212570 A1
(43) Pub. Date: Jul. 15, 2021

(54) Endoscopic Photoacoustic Probe

(71) Applicant: Universite Grenoble Alpes, Saint Martin D'heres (FR)

(72) Inventors: Guillaume Custillon, Echirolles (FR); Nicolas Dubois, Montbonnot Saint Martin (FR)

(73) Assignee: Universite Grenoble Alpes, Saint Martin D'heres (FR)

(21) Appl. No.: 15/734,048

(22) PCT Filed: Jun. 3, 2019
(86) PCT No.: PCT/FR2019/051294

§ 371 Date: Dec. 1, 2020

(30) Foreign Application Priority Data
Jun. 1, 2018 (FR) 1854804

(51) Int. Cl. A61B 5/00

(52) U.S. Cl. CPC A61B 5/0095; A61B 5/6852; A61B 2562/227; A61B 5/4528; A61B 5/4514

(57) ABSTRACT

The invention relates to a photoacoustic ultrasound probe (100), comprising:
a catheter (4),
an ultrasound sensor (5) arranged at a distal end (40) of the catheter,
at least one optical fibre (50) suited to being connected to a laser source, said optical fibre extending into the catheter up to the distal end (40),
said probe being characterised in that the catheter has an inclined distal portion (42) and/or a bevelled distal end (40).

### FIELD OF THE INVENTION

The present invention relates to an endoscopic photoacoustic probe.

### PRIOR ART

Medical imaging groups together means for acquiring and rendering images from different physical phenomena: magnetic resonance, reflection of ultrasonic waves, radioactivity, absorption of X-rays, etc. These technologies make it possible to view the physiology or the metabolism of the human body, to establish a precise diagnosis and to orientate therapeutic choices. However, they represent an imperfect imaging panoply: some information remains inaccessible or not very user-friendly (notably not being in real time), and irradiations are recognised as a major problem for patients and medical staff.

Only certain technologies are available in the operating theatre depending on the type of surgery. Orthopaedics requires the identification of bone structures and thus mainly resorts to the use of X-rays.

The use of intraoperative imaging has the aim of providing additional information with regard to damaged tissues, of increasing the precision of the operative procedure, of reducing the intervention time in a context of mini-invasive surgery, which makes it possible to increase the clinical benefits.

Arthroscopy is a mini-invasive surgical intervention during which a miniature camera is introduced into a joint via a punctiform incision and a miniature instrument is introduced through a second punctiform orifice. The surgeon may thus carry out a diagnostic act while assessing the anatomical structures of the articular cavity and a suitable therapeutic act thanks to the appropriate instrument. This surgical approach, performed on an out-patient basis, has revolutionised surgical practice by making it possible to operate without opening up and with a decrease in post-operative morbidity (decrease in hospitalisation time and functional readaptation period).

The joints of the knee and the shoulder quickly benefited from this surgical innovation. All or part of the anatomical structures constituting these joints may be altered during a traumatism or an osteoarthritis. An arthroscopy of the knee may thus concern the meniscuses (for example in view of an ablation), the cartilage (for example in view of a regularisation), the synovial membrane (for example for an adhesion excision), the resection of small cartilaginous or bony fragments (foreign bodies), or certain more important surgeries of the knee (ligamentoplasty or others). Similarly, and in a non-exhaustive manner, an indication of arthroscopy of the shoulder may be posed for the care of rotator cuff calcific tendinopathy, a sub-acromial conflict treated by carrying out a bursectomy and acromioplasty under arthroscopy, or instead to qualify, intraoperatively, a rupture of the rotator cuff.

During an arthroscopy, the visual exploration of the inside of the articular cavity is limited to the surface of the structures constituting the joint, with, as consequences, potential diagnostic or therapeutic limitations. Furthermore, since it involves an inaccessible zone, it does not lend itself to the injection of a contrast agent or a fluorescent agent to show up the vascularisation of the anatomical structures.

An endoscopic photoacoustic probe has the capacity of imaging anatomical structures in depth but also the vascularisation of said structures and thus provides additional information to the surgeon.

However, the configuration of a joint and the space available for the insertion of such a probe make the use of photoacoustics in arthroscopy difficult.

### DESCRIPTION OF THE INVENTION

An aim of the invention is to conceive an endoscopic photoacoustic probe suited for arthroscopy.

To this end, the invention proposes a photoacoustic ultrasound probe, comprising:
a catheter,
an ultrasound sensor arranged at a distal end of the catheter,
at least one optical fibre suited to being connected to a laser source, said optical fibre extending into the catheter up to the distal end,

said probe being characterised in that the catheter has an inclined distal portion and/or a bevelled distal end.

In the present text, the relative terms “proximal” and “distal” are understood respectively to be a part of the probe situated on the side of the operator who handles it, and of a part of the probe on the side of the body of the patient into which it is intended to be inserted.

According to an embodiment, the catheter has a distal portion inclined by an angle comprised between 10 and 30° with respect to a proximal portion of the catheter. According to an embodiment, the distal end is inclined by an angle comprised between 10 and 30° with respect to an axis of revolution of the distal part of the catheter.

In a particularly advantageous manner, the distal end of the catheter extends in a plane inclined by 30° with respect to the proximal portion of the catheter.

According to a preferred embodiment, the probe comprises several optical fibres arranged on either side of the ultrasound sensor.

According to an embodiment, the probe further comprises:
a control handle attached to the proximal portion of the catheter,
a connector suited to being connected to an ultrasound station,
an electrical connection cable extending between the connector and the handle,
a sheath comprising a first portion surrounding the catheter, made of a material suited to allowing the ultrasounds and the laser beam to pass, a second portion surrounding the handle and the cable up to the connector, and a fitting arranged between the first and the second portion, said fitting being removably attached to the handle.

According to a preferred embodiment, the first portion of the sheath is made of a polyamide-polyether block copolymer.

In an advantageous manner, said first portion has a constant thickness.

According to an embodiment, the thickness of the first portion is comprised between 0.4 and 0.6 mm.

Preferably, the second portion of the sheath has a diameter greater than that of the first portion.

In an advantageous manner, the fitting comprises a central opening for the passage of the catheter, the first portion being attached around said central opening of the fitting and the second portion being attached to a peripheral portion of the fitting.

Another object of the invention relates to an imaging system comprising an ultrasound station including a laser source, and an endoscopic photoacoustic probe such as described above connected to said station, in which the station comprises a processor configured to display an image of the vascularisation of an intra-articular zone of interest acquired by the probe.

In a particularly advantageous manner, the processor is configured to superimpose an ultrasound image of a meniscus and a photoacoustic image of the vascularisation of said meniscus.

### DESCRIPTION OF THE FIGURES

Other characteristics and advantages of the invention will become clear from the detailed description that follows, with reference to the appended drawings in which:

FIG. 1 is a perspective view of the catheter of a probe according to the invention;

FIG. 2 is a side view of said catheter;

FIG. 3 is a front view of the catheter;

FIG. 4 is a schematic diagram of the probe;

FIG. 5 illustrates the probe of FIG. 4 provided with a sheath;

FIG. 6 is a perspective view of the sheath alone;

FIG. 7 is a schematic view of a mounting for testing the photoacoustic probe;

FIG. 8 presents images of the vascularisation of a sheep meniscus acquired with the photoacoustic probe;

FIG. 9 illustrates in a schematic manner the vascularisation of the human meniscus according to the different zones;

FIG. 10 is a schematic view of a first mounting for qualitatively testing the sheath;

FIGS. 11A-11D are ultrasound images obtained thanks to the mounting of FIG. 10, respectively in the absence of a sheath, with a sheath made of PEBD, with a sheath made of PEBAX™ 2533 and with a sheath made of PEBAX™ 3533;

FIG. 12 is a schematic view of a second mounting for qualitatively testing the sheath;

FIG. 13 is a graph representing the overall attenuation (in dB) calculated by integration of the attenuations of FIG. 12 over all of the frequencies for the different materials tested;

FIGS. 14A and 14B are ultrasound images of a thick animal cartilage obtained by a probe respectively exempt of a sheath and protected by a sheath made of PEBAX™ 2533;

FIGS. 15A and 15B are ultrasound images of a thin cartilage obtained by a probe respectively exempt of a sheath and protected by a sheath made of PEBAX™ 2533.

Identical reference signs from one figure to the other designate identical elements or elements fulfilling the same function.

### DETAILED DESCRIPTION OF EMBODIMENTS OF THE INVENTION

The endoscopic photoacoustic probe forms part of an imaging system comprising an ultrasound station to which the probe is connected.

The station comprises an ultrasound control electronic, a laser source, a processor suited to processing the images acquired by the probe, a viewing screen and a keyboard making it possible to enter data and to annotate the images.

During an operation under arthroscopy of the shoulder or of the knee, the endoscopic photoacoustic probe is inserted into the joint using the instrumental route. The probe is still used with the arthroscope which uses the other route. The surgeon brings the distal part of the probe into the zone of interest with the aid of the arthroscope and inspects the anatomical structures in this zone by orienting the probe towards them.

The photoacoustic probe comprises at one and the same time an ultrasound sensor which makes it possible to take ultrasound images in the joint and one or more optical fibres connected to the laser source to acquire photoacoustic images, which may be combined or not with the ultrasound images. Indeed, the excitation by a laser beam of an anatomical structure containing haemoglobin generates an ultrasonic wave which is recorded by the ultrasound sensor to provide a photoacoustic image. The ultrasound sensor may further emit an ultrasonic wave and collect the ultrasonic wave reflected by the anatomical structures to provide an ultrasound image. The photoacoustic image may be superimposed on the ultrasound image.

The frequency of the ultrasounds is advantageously comprised in the range extending from 5 to 60 Mhz.

The wavelength of the laser beam is advantageously comprised between 500 and 1200 nm.

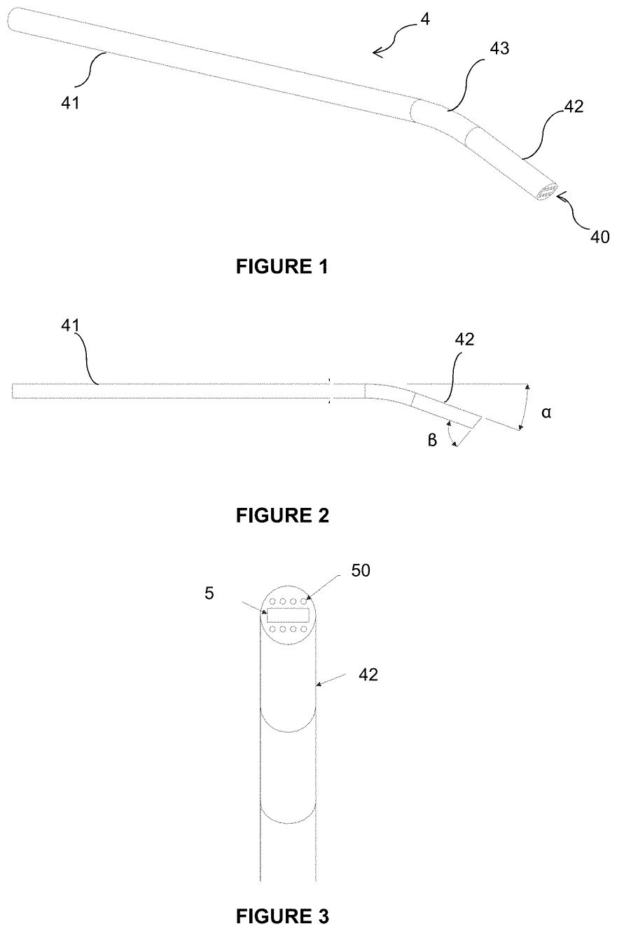

FIGS. 1 to 3 are views of a catheter of a probe according to the invention.

The catheter 4 comprises an ultrasound sensor 5 and at least one optical fibre 50 which extends along the catheter up to the distal end 40. The distal end of the catheter comprises a lens facing said optical fibre.

Preferably, the catheter comprises several optical fibres, for example from 6 to 12 optical fibres, in order to increase the excitation light signal.

The ultrasound sensor advantageously comprises a multi-element transducer arranged in the form of a linear array, for example 15 to 25 elements in number.

In an advantageous manner, the ultrasound sensor 5 is arranged at the distal end 40 of the catheter. The optical fibres 50 are preferably arranged on either side of the ultrasound sensor.

The catheter has a length generally comprised between 10 and 20 cm and a diameter of several millimetres, for example 4 mm. The catheter may be made of stainless steel or titanium.

The catheter has an inclined distal portion 42 and/or a bevelled distal end 40. “Inclined distal portion” is taken to mean that the distal portion 42 of the catheter is inclined with respect to the proximal portion 41 of the catheter (angle α in FIG. 2), the distal and proximal portions, which are straight, being connected by a curved portion 43. “Bevelled distal end” it taken to mean that the plane of the distal end 40 is inclined with respect to the axis of revolution of the distal portion 42 of the catheter (angle ß in FIG. 2). The angles of inclination α, ß are typically of the order of some ten to some thirty degrees and may be combined.

Thus, a configuration of the catheter with an inclination of the distal end of the catheter of 30° with respect to the proximal portion is particularly advantageous in arthroscopy, because it makes it possible to better adapt to the geometry of the joint to inspect. For example, the catheter may have a distal portion inclined by 10° with respect to the proximal portion of the catheter, and an end bevelled by an angle of 20° with respect to the axis of revolution of the catheter.

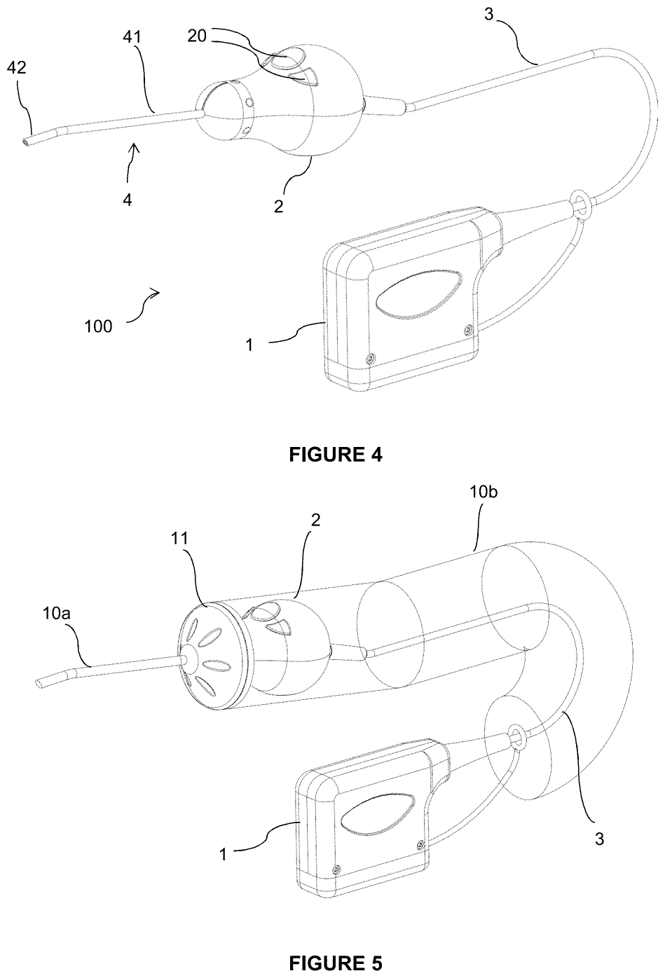

FIG. 4 is a schematic diagram of the probe.

The probe 100 comprises a connector 1 through which it may be connected to the ultrasound station (which is not represented).

The connector 1 is connected to a control handle 2 through an electrical connection cable 3. The control handle is the member that the user holds in his hand during the intervention. The handle comprises one or more buttons 20 that the user can press for example to browse or to validate the acquisition of an image or a video.

The proximal portion 41 of the catheter 4 is attached to the handle 2.

According to an advantageous embodiment, the probe is protected by a sterile sheath which covers it entirely, from the distal end of the catheter up to the connector which connects it to the ultrasound station. The sheath is in one piece and only has an opening at the level of the connector.

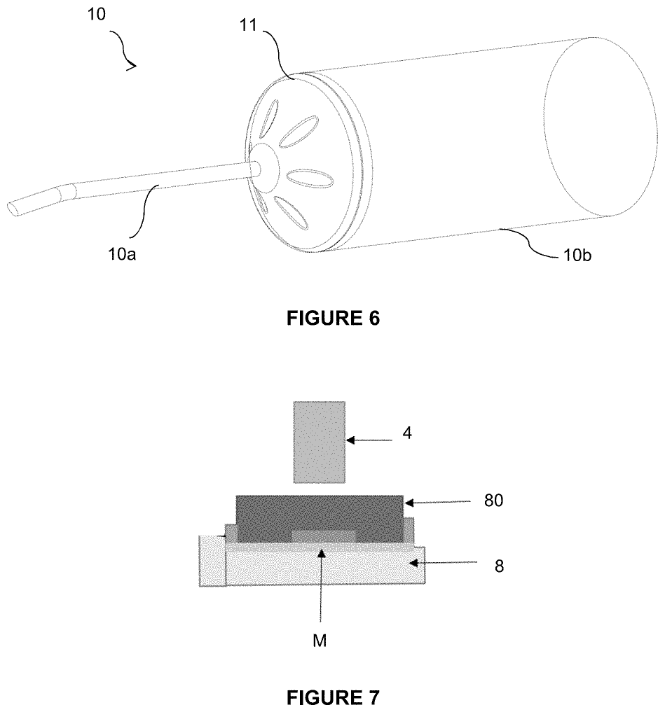

FIG. 5 is a diagram of the endoscopic photoacoustic probe equipped with such a sheath, and FIG. 6 represents the sheath alone.

The sheath 10 has a first part 10a suited to surrounding the catheter, and a second part 10b, wider, surrounding the handle and the connection cable. The dimensions of the sheath are chosen to adapt to the shape and to the dimensions of the probe. Generally speaking, the first and the second part each have a cylindrical shape of circular section.

The first part 10a hugs the shape of the catheter 4 as closely as possible in order not to increase excessively the bulk of the catheter, a slight clearance being arranged to facilitate the slipping of the sheath onto the catheter. For example, for a sheath of 0.5 mm thickness and a catheter of 4 mm diameter, the outer diameter of the first portion is of the order of 5.4 mm. The first portion 10a of the sheath is closed at its distal end, including facing the lens. The first portion 10a is sufficiently flexible given its material and its thickness to adapt to the shape and to the curvature of the catheter if need be.

The first and the second part of the sheath are connected by a fitting 11 thanks to which the sheath may be removably attached to the handle of the probe.

The fitting is typically made of a thermoplastic material.

The first portion 10a of the sheath is made of a material suited to allowing the laser beam and the ultrasound signal to pass with a sufficiently low attenuation so that the imaging can be carried out with a sufficient resolution. The choice of a material suited for this function will be described in detail hereafter. Preferably, the first portion of the sheath is made of a polyamide-polyether block copolymer sold notably under the denomination PEBAX™. Preferably, PEBAX™ 2533 is preferred, PEBAX™ 3533 being satisfactory but less preferred. The thickness of the first portion of the sheath is chosen sufficiently thin so as not to bring about a too important attenuation of the ultrasounds. For example, the thickness of this first portion is comprised between 0.4 and 0.6 mm, in a preferred manner 0.5 mm. This thickness is advantageously constant over the entire length of the first portion.

The second portion 10b of the sheath may be made of the same material as the first portion or made of another material, this second portion not being intended to allow ultrasounds to pass. The second portion does not necessarily comprise means of attachment to the connector. It is in fact sufficiently long to receive the probe up to the non-sterile zone of the operating site, even if it is simply slipped around the connection cable.

The fitting 11 has a central opening for the passage of the catheter 4, around which it extends radially, the first portion 10a of the sheath being attached to a central portion of the fitting, around said opening 110, and extends from the distal end of the fitting; the second portion 10b of the sheath is attached to a peripheral portion of the fitting and extends from the proximal end of the fitting. The first and the second portions 10a, 10b are attached in a leak tight manner to the fitting 11 by any appropriate means, for example by bonding on the fitting or by over-moulding of the fitting.

The fitting 11 and the handle 2 advantageously have mutual reversible fastening together means. According to an embodiment, the fastening together is carried out by a quarter turn mechanism. In an alternative manner, the fastening together may be carried out by snap fitting.

The material(s) forming the two portions of the sheath and the fitting may be sterilised by ionising radiation without degrading. Preferably, the sterilisation is carried out by exposure to ethylene oxide followed by exposure to y rays.

The sheath 10 being removable vis-à-vis the remainder of the probe and entirely covering the part of the probe situated in the operating field, it may be sterilised separately and makes it possible to procure a sterile endoscopic photoacoustic device without risking damaging the functional components of the probe. Typically, the sheath is sterilised before being packaged in a packaging suited to preserving the sterility and is only extracted from this packaging to be put in place on the probe.

The modalities for choosing the material of the first portion of the sheath will now be described.

The inventors have tested different materials transparent to ultrasonic waves and have qualitatively and quantitatively evaluated them to verify their capacity to allow the ultrasound signal to pass with a sufficiently low attenuation. In so far as the problem of the material of the sheath is posed mainly for the ultrasonic signal, the sheath was tested for an ultrasound probe without implementation of laser excitation.

FIG. 10 is a schematic diagram of a first mounting for a qualitative test.

The ultrasound sensor 5 of the probe is arranged facing a wall 6 made of PVC, the assembly being immersed in water.

A first ultrasound image is acquired without the sheath, then a sheath 10a constituted of each of the materials to test is slipped onto the catheter 4, and a respective ultrasound image is acquired.

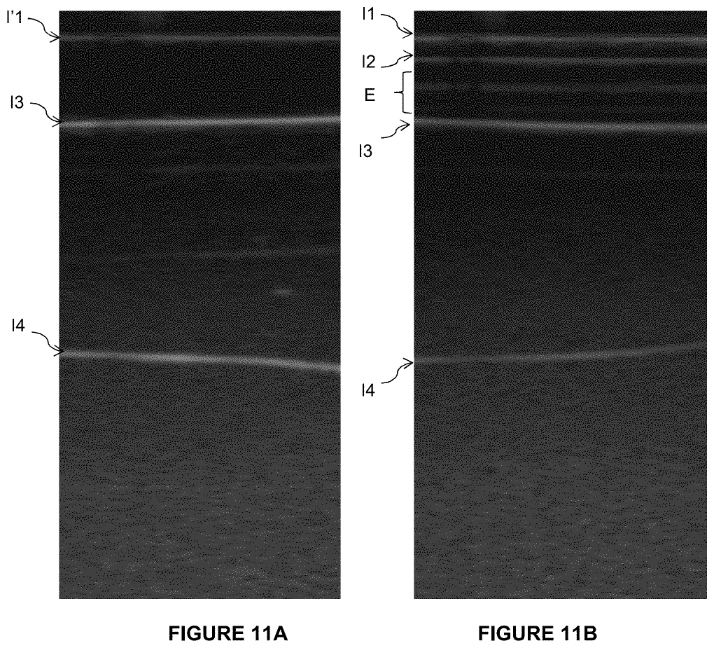

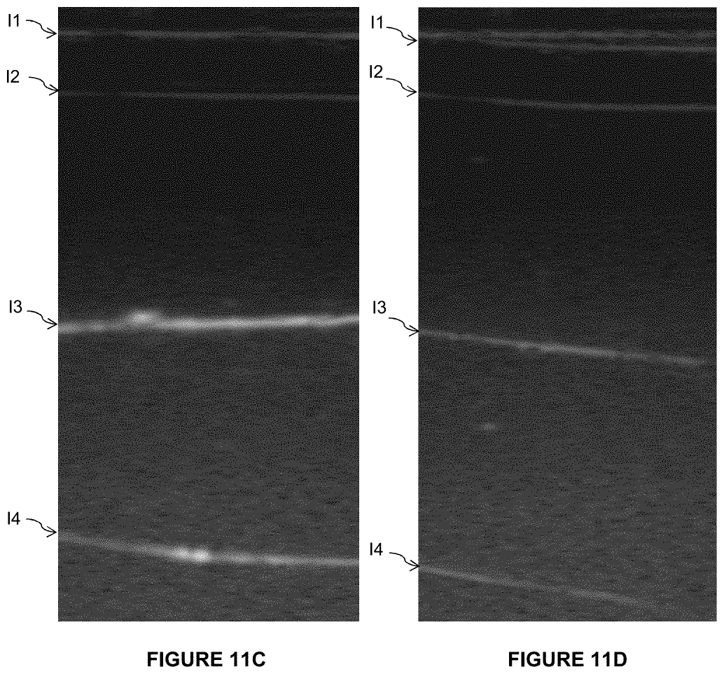

The results presented in FIGS. 11A-11D are respectively obtained without a sheath, with a sheath made of PEBD, with a sheath made of PEBAX™ 2533 (preferred material) and made of PEBAX™ 3533. The white lines designated by l1, l2, l3 and l4 correspond respectively to the interface between the lens and the sheath, the interface between the sheath and water, the interface between water and PVC (surface of the wall facing the probe) and the interface between PVC (surface of the wall opposite to the probe) and water. In the case of FIG. 11A, in the absence of a sheath, the interface between the lens and water is designated by l′1.

A first criterion for qualification of a material envisaged for the sheath is the visibility of these interfaces. It is sought to obtain a visibility as close as possible to the visibility obtained without a sheath. The loss of visibility of the interfaces 61, 62 of the PVC wall is due to the absorption of ultrasounds in the sheath and to the energy reflected by the inner 101 and outer 102 surfaces of the sheath.

Furthermore, the potential lines designated by E correspond to parasitic interfaces due to a cavity phenomenon in the thickness of the sheath. This phenomenon is caused by a difference in acoustic impedance between the sheath and water. Hence, the ultrasounds travel back and forth multiple times between the outer surface of the sheath and the probe, each back and forth journey producing an echo which is translated by a horizontal line on the image. Such parasitic interfaces adversely affect the quality of the image. These parasitic echoes are all the more problematic given that in the targeted intra-articular application the probe must be very close to the tissues to ultrasound, or even in contact therewith, which implies that potential parasitic echoes are superimposed on the anatomical structures to observe.

The presence and the number of parasitic echoes thus constitutes a second criterion for qualification of the material. Such echoes must be avoided or at least minimised.

It may be observed in these images that the sheath made of PEBD produces parasitic echoes, even though the expected interfaces have good visibility. The sheath made of PEBAX™ 2533, which is the most high-performance material, does not generate parasitic echoes while procuring clearly visible interfaces. PEBAX™ 3533 also appears as a relatively satisfactory material.

The table below recapitulates the tested materials and their classification in terms of number of parasitic echoes and visibility of interfaces.

| | Number of parasitic | Visibility of the |
|---|---|---|
| Sheath material | echoes | interfaces |
| Without sheath | 0 | +++ |
| PA12 | 4 | + |
| PEBD | 2 | ++ |
| PVC | 2 | + |
| Silicone | 0 | − |
| TPU95 | 2 | −− |
| TPU98 | 3 | −− |
| PEBAX ™ 2533 | 0 | ++ |
| PEBAX ™ 3533 | 0 | + |
| PEBAX ™ 4533 | 0 | − |

Another study was conducted to evaluate quantitatively the attenuation caused by the different materials envisaged for the sheath.

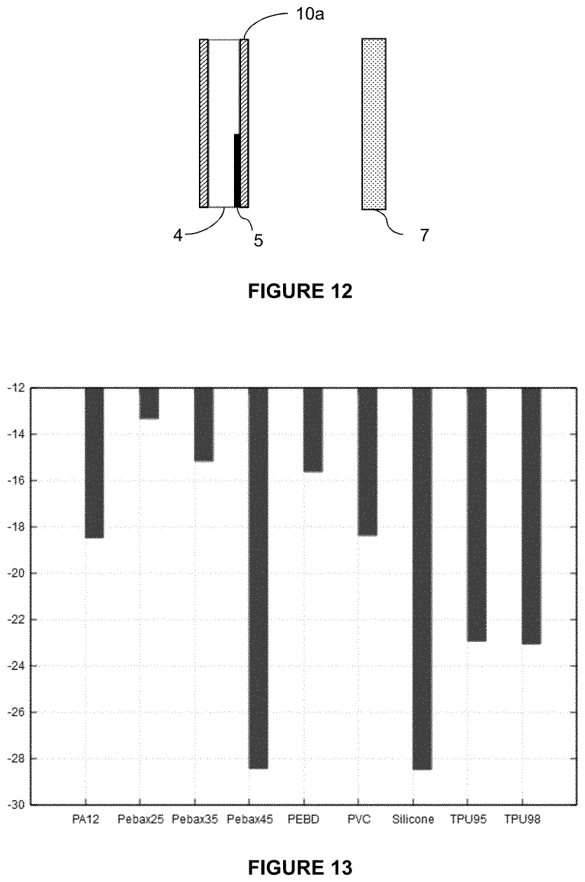

FIG. 12 is a schematic diagram of a second mounting for a qualitative test.

The ultrasound sensor 5 of the probe is arranged facing a wall 7 made of metal, which is a good reflector of ultrasounds, the assembly being attached by a bench to maintain a constant distance between the probe and the metal wall.

A first ultrasound image is acquired without the sheath, then a sheath 10a constituted of each of the materials to test is slipped onto the catheter 4, and a respective ultrasound image is acquired. An attenuation calculation is then performed.

FIG. 13 is a graph representing the overall attenuation (that is to say integrated over the whole of the frequency range of interest, which is comprised between 10 and 15 MHz) (in dB) for the different materials tested.

It may be observed that silicone and PEBAX™ 4533 have strong absorption, and are thus not preferred, even though they do not generate parasitic echoes. PEBAX™ 2533 is the best candidate, with an absorption of around 13 dB, followed by PEBAX™ 3533 (15 dB) and PEBD (15.5 dB). However, PEBD will not be retained on account of the parasitic echoes that it generates.

In conclusion, PEBAX™ 2533 is the tested material that has the least attenuation. In addition, it is important to note that the sheaths made of PEBAX™ tested were thicker (around 2 mm thickness) than the others (around 0.8 mm). Thus, in conditions of identical thickness, a sheath made of PEBAX™ 2533 would have even less attenuation than the others.

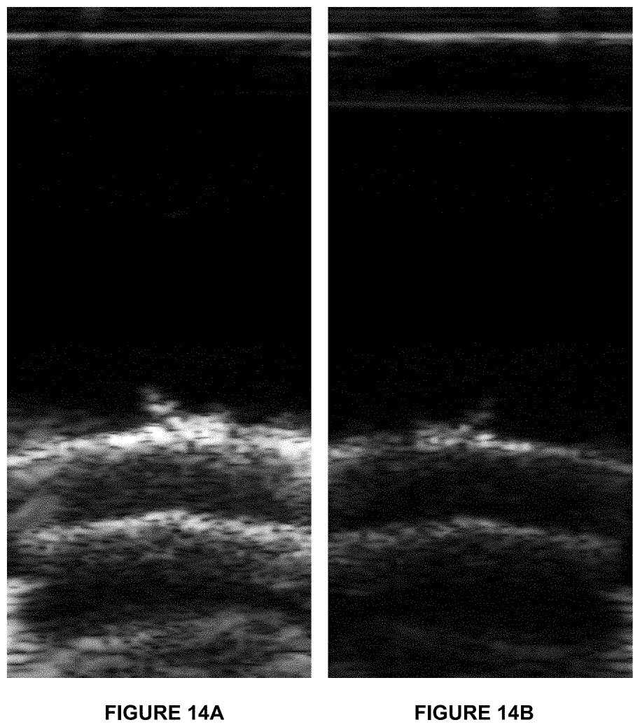

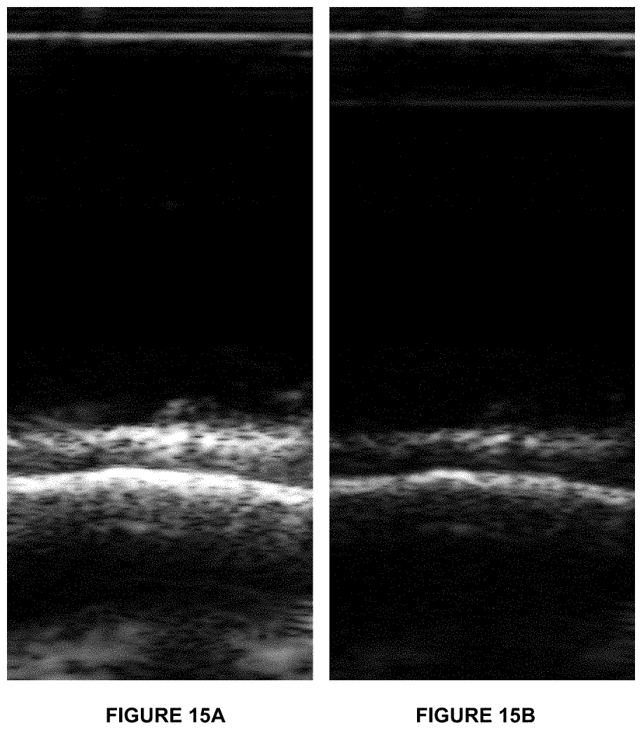

To validate that PEBAX™ 2533 indeed corresponds to the expectations, cartilage images were taken while maintaining the probe fixed on animal anatomical parts. All the images were recorded with the same adjustments of parameters. Thick (that is to say of around 2.6 mm thickness) (cf. FIGS. 14A-14B) and thin (that is to say of around 1.6 mm thickness) (cf. FIGS. 15A-15B) cartilage images were recorded (visible in the lower part of the images). The interfaces delimiting the cartilage are still visible with the sheath made of PEBAX™ 2533.

When the probe is bare in water, the interfaces are very shiny and saturate the image: it would be necessary to decrease the gain to observe it correctly. With the sheath made of PEBAX™ 2533, the saturation disappears, as if the gain had been decreased to an acceptable level.

From an acoustic viewpoint, PEBAX™ 2533 thus appears as the best material: its acoustic impedance is close to that of water (no parasitic echoes due to the cavity phenomenon) and it absorbs little ultrasounds. Consequently, the images obtained through this sheath have sufficient quality for a viewing in depth of the anatomical structures of interest, while ensuring the sterility of the probe.

In addition, PEBAX™ 2533 is a translucid material, through which the inside of the probe may be seen, notably the position of the ultrasound sensor on the arthroscopic images, which makes it possible to facilitate the use of the probe by a surgeon. This material is thus transparent to the laser beam and thus enables the emission of the laser beam and the emission/reception of ultrasound waves.

FIG. 7 is a diagram of a mounting for testing the photoacoustic probe. A fresh sheep meniscus (designated by the mark M), still having traces of haemoglobin, was placed on a sample holder 8 and fixed in agarose 80. The photoacoustic probe (only the catheter 4 is represented) was put in place above the sample and photoacoustic images of the meniscus were acquired. The wavelength of the laser source was 532 nm, and the ultrasonic frequency 15 MHz.

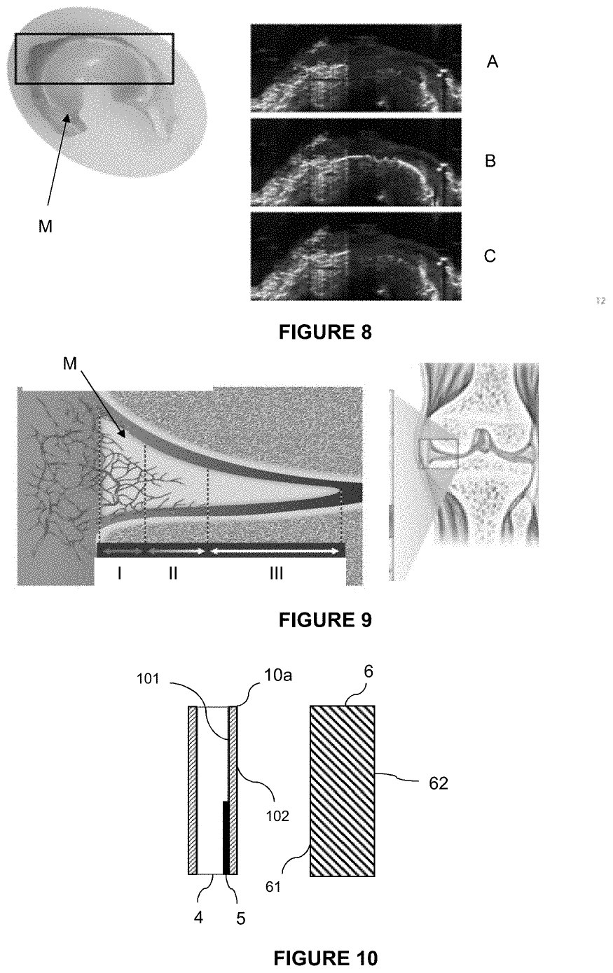

FIG. 8 shows images of the vascularisation of a sheep meniscus acquired with the photoacoustic probe. The ultrasound image is an image averaged over the whole thickness of the meniscus, corresponding to the framed zone in the photograph of the meniscus. In image A, it is the maximum of the values of the photoacoustic signal over the thickness of the meniscus which has been transferred onto the ultrasound image.

In image B, it is the average of the values of the photoacoustic signal over the thickness of the meniscus which has been transferred onto the ultrasound image.

In image C, it is the values of the photoacoustic signal over the thickness of the meniscus above a threshold which have been transferred onto the ultrasound image, in order to eliminate the photoacoustic signal outside of the volume (corresponding to noise).

These images show the presence of a photoacoustic signal on the outer zone of the meniscus, probably coming from the haemoglobin contained in the vessels of the meniscus. It is in this outer zone that there is expected to have the most vascularisation, as shown in FIG. 9.

Meniscuses are semilunar fibrocartilages interposed between the femoral condyles and the tibial plateau. They have an anchorage by their anterior and posterior horns at the level of the pre- and retro-spinal surfaces of the tibia, as well as a circumferential capsuloligamentary anchoring by their peripheral edge. These are moveable and deformable structures undergoing considerable compressive, shear and torsional forces which can lead to various lesions. Their function is henceforth known: stress distribution, stabilisation of the knee on the anterior translation, nutrition and lubrication of the joint and proprioception.

Lesions of the meniscuses are frequent and affect the whole of the population. They may be traumatic and/or degenerative. They cause pain, blockages and articular effusions. Their treatment is simple and consists in a resection or a repair of the meniscus.

The result in the medium and long term of a meniscectomy, which consists in an ablation of the meniscus (with a possible development of secondary osteoarthritis), and the results of reconstructions of the anterior cruciate ligament incite practitioners to preserve the meniscus. Indeed, when it is removed, its absence contributes to an acceleration of the degradation of the joint, and to the onset of early osteoarthritis, in particular in sports enthusiasts (skiing, foot races, football, etc.), including in young people. The preservation of the meniscus makes it possible to prevent degradation of the knee, and to delay as long as possible the implantation of a total knee prothesis. At the present time, the only technology proposed in the event of lesion of the meniscus is the suture, yet the literature shows a failure rate of up to 43% (Pujol et al, “Amount of meniscal resection after failed meniscal repair”, Am. J. Sports Med, 2011). One of the major criteria for the establishment of a successful prognosis is the visualisation of the blood capillaries (constituting a micro-vascularisation) at the level of the injured zone. This micro-vascularisation is not at the present time detectable under operating conditions, or in pre-operative imaging. The lesions being in more than 50% of cases situated in the uncertainty zone, the decision of the surgeon leans in the majority of cases towards an ablation rather than towards a repair with an uncertain outcome. Furthermore, due to the high cost of suture buttresses, the quantities stockpiled in operating theatres and the use thereof are subject to controlled use.

FIG. 9 is a sectional view schematically showing the different micro-vascularisation zones of the meniscus. Meniscal vascularisation takes place from the periphery of the meniscus (meniscal wall) whereas the free edge of the meniscus is not vascularised. It is possible to distinguish three zones: zone I (also called red zone) is the most vascularised; zone II (also called red-white zone) is moderately vascularised; finally, zone III (also called white zone) is not vascularised. In practice, a suture carried out in zone I has good changes of healing perfectly; on the other hand, a suture carried out in zone II has random chances of healing. Finally, a suture carried out in zone III is doomed to failure on account of the absence of vascularisation.

Meniscal repair is possible if the meniscal lesion is seated near to the peripheral insertion of the meniscus and if it is situated in the longitudinal vertical plane. This suture is performed according to the same general modalities as meniscal regularisation. Technically, putting in place a resorbable anchor makes it possible to secure the suturing thread. Meniscal repair is especially indicated in young subjects and subjects having presented a meniscal lesion following rupture of the anterior cruciate ligament. Meniscal suture on a stabilised knee will then enable a favourable evolution and healing in the best conditions not only of the cruciate ligament but also of the meniscus brace.

The endoscopic photoacoustic probe procures for the surgeon an intraoperative decision aid tool making it possible to determine, with a high level of certainty, if, as a function of micro-vascularisation, suture may be carried out with success.

Photoacoustics allies in fact two advantages: on the one hand the possibility of specifically exciting haemoglobin (characteristic of the target that are the blood capillaries), and on the other hand the possibility of having available sufficient sensitivity to detect these excitations in the depth of the anatomical structure considered (for example meniscus in fibrocartilage). The use of the probe is a diagnostic act complementary to current practices which is part of the progress of the surgical act because it provides additional information to orient the decision between repair or ablation of the meniscus.

For the same reasons, the endoscopic photoacoustic probe also has an interest in the treatment of lesions of the rotator cuff in the shoulder. Indeed, vascularisation of the rotator cuff is one of the key elements in tendon healing after repair of lesions.

1. A photoacoustic ultrasound probe, comprising:
a catheter,
an ultrasound sensor arranged at a distal end of the catheter, and
at least one optical fibre configured for being connected to a laser source, said optical fibre extending into the catheter up to the distal end,
wherein the catheter has at least one of an inclined distal portion and a bevelled distal end.

2. The photoacoustic ultrasound probe of claim 1, wherein the catheter has a distal portion inclined by an angle comprised between 10 and 30° with respect to a proximal portion of the catheter.

3. The photoacoustic ultrasound probe of claim 1, wherein the distal end is inclined by an angle comprised between 10 and 30° with respect to an axis of revolution of the distal part of the catheter.

4. The photoacoustic ultrasound probe of claim 2, in which the distal end (40) of the catheter extends in a plane inclined by 30° with respect to the proximal portion (41) of the catheter.

5. The photoacoustic ultrasound probe f claim 1, further comprising several optical fibres arranged on either side of the ultrasound sensor.

6. The photoacoustic ultrasound probe of claim 1, further comprising:
a control handle attached to the proximal portion of the catheter,
a connector configured for being connected to an ultrasound station,
an electrical connection cable extending between the connector and the handle, and
a sheath comprising a first portion surrounding the catheter, made of a material suited to allowing the ultrasounds and the laser beam to pass, a second portion surrounding the handle and the cable up to the connector, and a fitting arranged between the first and the second portion, said fitting being removably attached to the handle.

7. The photoacoustic ultrasound probe of claim 6, wherein the first portion is made of a polyamide-polyether block copolymer.

8. The photoacoustic ultrasound probe of claim 6, in wherein the first portion has a constant thickness.

9. The photoacoustic ultrasound probe of claim 6, wherein the thickness of the first portion is comprised between 0.4 and 0.6 mm.

10. The photoacoustic ultrasound probe of claim 6, wherein the second portion has a diameter greater than a diameter of the first portion.

11. The photoacoustic ultrasound probe of claim 6, wherein the fitting comprises a central opening for the passage of the catheter, the first portion being attached around said central opening of the fitting and the second portion being attached to a peripheral portion of the fitting.

12. An imaging system comprising an ultrasound station including a laser source, and an endoscopic photoacoustic probe according to claim 1 connected to said ultrasound station, wherein the ultrasound station comprises a processor configured to display an image of the vascularisation of an intra-articular zone of interest acquired by the probe.

13. The imaging system of claim 12, wherein the processor is configured to superimpose an ultrasound image of a meniscus and a photoacoustic image of the vascularisation of said meniscus.
